(19) United States
(12) Patent Application Publication
(10) Pub. No.: US 2015/0123432 A1
(43) Pub. Date: May 7, 2015

(54) Surgeon's Assistant

(71) Applicant: Robert Carl Ray, Morehead, KY (US)

(72) Inventor: Robert Carl Ray, Morehead, KY (US)

(21) Appl. No.: 14/513,381

(22) Filed: Oct. 14, 2014

Related U.S. Application Data
(60) Provisional application No. 61/898,937, filed on Nov. 1, 2013.

(51) Int. Cl. A61B 19/00

(52) U.S. Cl. CPC A61B 19/28
USPC 297/188.01

(57) ABSTRACT

Surgeons everywhere have to stand erect for very long periods of time, whether that be for an entire day of surgeries or for a single, complicated surgery, many of whom develop their own muscular-skeletal disorders. Most if not all Surgeons suffer from chronic back, cervical, lumbar, and neck pain due to their unforgiving postures while performing surgeries and especially during lengthy surgeries. Their arms are often times extended, which multiplies and exacerbates this problem as well as their head, neck and upper body, all of which tax the spinal cord and muscular support systems.

Nothing else, no other device or supporting structure exists in this space, market and surgical suite.

This invention takes the load off the surgeon and puts the load onto a mechanical device and places the surgeon into the best possible posture and comfort for which to conduct surgery and rest his own body while performing the surgery. It is a whole body ergonomic system for Surgeons.

### THE VERSION OF THE INVENTION DISCUSSED HERE INCLUDES

1. Build a multi-articulating metal structure to accommodate any body type, the main frame of which will support all of the weight, weight of the structure, weight of the surgeon, weight of any and all surgical devices accoutrements and Appurtenances necessary to conduct the given surgery and or surgical procedure.

2. Include articulating and positional Torso, Upper Body, arms and arm, Head, Neck and Face rests

3. Include articulating and positional hand and hand rests; wrist wrest and finger rests if needed.

4. Include articulating and positional Lower Body, leg and leg rests

5. Include articulating and positional foot and foot rests, buttock rests can also be integrated into the system.

6. Build the device to accept add on components at the surgeons hand/surgical field level

7. Build the device to accept add on components at the surgeons face/eye level

8. Build the device to allow the surgeon to heat and or cool themselves

9. Build the device to operate either manually or electrically, have electrical outlets built into the device for each use of electrically operated tools, equipment and machinery.

10. Build the device to be sanitary

11. Build the device to meet Joint commission accreditation standards

12. Build the device to be portable, man movable, electrically creep capable for fine motion and motor function and movement of the surgeon.

13. Build the device to be coated with a special coating that will not peel, chip or fall off into the surgical field.

14. Build the device to accept all other future proof of surgical tools, equipment and devices as add on devices to the original design structure

15. Build the device to be one man mobile and on wheels.

16. Build the device to have fail safes built into the structure.

17. Build the device to have locking pit pins at areas of articulation for backup.

18. Build the device to have hydraulics where needed.

19. Build the device to have electrical motors for movement as needed.

20. Build the device for compactness, folding and storage.

### DRAWING EXPLANATIONS AND ILLUSTRATIONS

(FIGS. 1-15):

FIG. 1 This is the Multi-Axes Head/Face Rest. This component can articulate in the traditional 3 Axes x,y,z, but can also rotate, extend, contract, roll, pitch and yaw; to accommodate any users anthropometric dimensions. It can also accommodate any attachment to assist the surgeon in the performance of his task, some examples are microscopes, magnification, lighting, instrument holders and or bays. The underside of this rest has integrated high intensity LED lighting to light the surgical site. The rest is made from ridged metal or carbon fiber but cushioning is provided for facial comfort. It will be built with all anthropometric considerations from 0-100 percentile of the population.

FIG. 2 This is the Axes pin that allows transverse motion and articulation of the Head/Face Rest, it will lock into place with at least 2 redundant positive locking mechanisms to ensure Surgeon and Patient safety. It too will be made from ridged metal substances or carbon fiber.

FIG. 3 This depicts an add on surgical device such as a magnification glass or some other device to assist the surgeon. The add on devices are limitless.

FIG. 4 This is the Multi-Axes articulating arm rest for the surgeon. This component can articulate in the traditional 3 Axes x,y,z, but can also rotate, extend, contract, roll, pitch and yaw; to accommodate any users anthropometric dimensions. It can also accommodate any attachment to assist the surgeon in the performance of his task, some examples are microscopes, magnification, lighting, instrument holders and or bays. It will also accommodate a Joy Stick at the end to operate other functions of the total machine, such as; up/down movement, in/out, left/right, and whatever other functions for which it is programmed to accomplish. The rest is made from ridged metal or carbon fiber but cushioning is provided for arm/forearm/wrist/hand comfort. It can swing out of the way when necessary and will be built with all anthropometric considerations from 0-100 percentile of the population. The underside too, could be lighted with LED lighting.

FIG. 5 This is the Joy Stick on both the left and right arm rests that can be programmed for a variety of functions for the locomotion of the entire machine. It will be made from hospital grade components to eliminate pathogen transmission.

FIG. 6 This is the Multi-Axes articulating arm rest for the surgeon. This component can articulate in the traditional 3 Axes x,y,z, but can also rotate, extend, contract, roll, pitch and yaw; to accommodate any users anthropometric dimensions. It can also accommodate any attachment to assist the surgeon in the performance of his task, some examples are microscopes, magnification, lighting, instrument holders and or bays. It will also accommodate a Joy Stick at the end to operate other functions of the total machine, such as; up/down movement, in/out, left/right, and whatever other functions for which it is programmed to accomplish. The rest is made from ridged metal or carbon fiber but cushioning is provided for arm/forearm/wrist/hand comfort. It can swing out of the way when necessary and will be built with all anthropometric considerations from 0-100 percentile of the population. The underside too, could be lighted with LED lighting. It too will be made from ridged metal substances or carbon fiber and will be built with all anthropometric considerations from 0-100 percentile of the population.

FIG. 7 This illustrates both the elongation and contraction capabilities of the arm rests. Both arm rests will have at least a double redundant positive locking system for Surgeon and Patient Safety. It too will be made from ridged metal substances or carbon fiber and will be built with all anthropometric considerations from 0-100 percentile of the population.

FIG. 8 This illustrates both the elongation and contraction capabilities of the arm rests. Both arm rests will have at least a double redundant positive locking system for Surgeon and Patient Safety. It too will be made from ridged metal substances or carbon fiber and will be built with all anthropometric considerations from 0-100 percentile of the population.

FIG. 9 This is the Axes pin that allows transverse motion and articulation of the Upper Torso Rest, it will lock into place with at least 2 redundant positive locking mechanisms to ensure Surgeon and Patient safety. It too will be made from ridged metal substances or carbon fiber and will be built with all anthropometric considerations from 0-100 percentile of the population.

FIG. 10 This illustrates the cushioned seat that will allow the surgeon to rest when necessary or as needed or to better position himself for his tasks. It will be fully articulating in all Axes. It too will be made from ridged metal substances or carbon fiber and will be built with all anthropometric considerations from 0-100 percentile of the population.

FIG. 11 this illustrates the Left cushioned leg rests, this component can articulate in the traditional 3 Axes x,y,z, but can also rotate, extend, contract, roll, pitch and yaw; to accommodate any users anthropometric dimensions. The rest is made from ridged metal or carbon fiber but cushioning is provided for facial comfort. It will be built with all anthropometric considerations from 0-100 percentile of the population.

FIG. 12 this illustrates the Right cushioned leg rests, this component can articulate in the traditional 3 Axes x,y,z, but can also rotate, extend, contract, roll, pitch and yaw; to accommodate any users anthropometric dimensions. The rest is made from ridged metal or carbon fiber but cushioning is provided for facial comfort. It will be built with all anthropometric considerations from 0-100 percentile of the population.

FIG. 13 This illustrates how both leg rests will have at least Two redundant positive locking safety devices for Surgeon and Patient Safety.

FIG. 14 this illustrates the weighted base, the weight and length of the legs will be engineered to accommodate Surgeons Anthropometric weights from 0 percentile to 100 percentile, the legs could extend or contract for additional weight support, the design of which will accommodate for any operating table configuration. The base will be on wheels for mobility and its locomotion can be either manually positioned or positioned via the Joy Sticks either of which are customer dependent. It too will be made from ridged metal substances or carbon fiber or both.

FIG. 15. This is the Axes pin that allows transverse motion and articulation of the Lower Torso Rest, it will lock into place with at least 2 redundant positive locking mechanisms to ensure Surgeon and Patient safety. It too will be made from ridged metal substances or carbon fiber and will be built with all anthropometric considerations from 0-100 percentile of the population. This component can articulate in the traditional 3 Axes x,y,z, but can also rotate, extend, contract, roll, pitch and yaw; to accommodate any users needs or requirements.

### RELATIONSHIP BETWEEN THE COMPONENTS

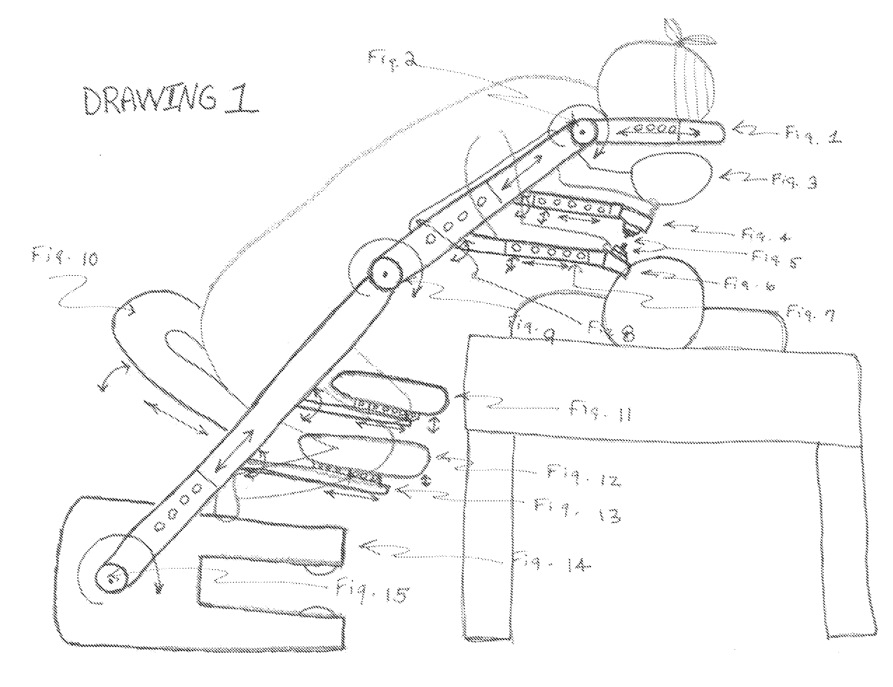

Items 1-20 (20-100 reserved or additional modifications) and FIGS. 1-15 are integrated into the device at both the design and build stage. To accommodate current surgical instruments and future proofed to accept those devices in the future.

### How The Invention Works

None of the steps can work individually, they all must be employed and built to work as a system.

### How To Make The Invention

The invention can be made in any capable machine, manufacturing or metal shop.

All elements are necessary to accommodate the current and future needs of a Surgeon.

The 1-20 elements can be shuffled, but all must eventually be integrated into the main supporting structure of the device itself.

### How To Use The Invention

The person would simple enter into or onto the device, position the x, y, z planes of the verticals, horizontals, in and out of the arms, legs and body supports; as well as the head support allowing the surgeon to perform surgeries in an almost zero gravity environment. This is achieved by taking all of the load off the surgeon and putting it onto the device, whereby the surgeons own body weight and the effects of gravity are zero, allowing the surgeon to perform delicate, intricate surgeries in a painless posture.

Additionally: any person doing work with their hands can use this device, dentists and other types of medical professionals.

1. As stated above, surgeons everywhere have to stand erect for very long periods of time, whether that be for an entire day of surgeries or for a single, complicated surgery. Most if not all Surgeons suffer from chronic spine, back and neck pain due to their unforgiving postures while performing surgeries. Their arms are often times extended, which exacerbates this problem as well as their head, neck and upper body, all of which tax the spinal cord and muscular support systems. The invention claimed here solves this problem.
This invention allows the Surgeon to enter into a mechanical/electrically operated system that supports the entire weight of the surgeon, arms, legs; and allows the surgeon to place himself into the best possible ergonomically relaxed posture they can be in to conduct the surgery. This device takes all of the load and effects of weight and gravity off the surgeon thus allowing the surgeon to have much longer surgeries without the surgeon himself from becoming exhausted, and suffer from his own muscular trauma.
Nothing no other device or supporting structure exists in this space, market and surgical suite, nothing else exists.
This invention is an improvement on what currently exists because; Nothing else exists. So surgeons worldwide who suffer daily from Muscular Skeletal trauma themselves and cumulative trauma that impairs the surgeon and ultimately impairs the patient, can be freed from their pain, so that they can give the patient the best possible patient care possible, without regard to what ails the surgeon.
This invention takes the load off the surgeon and puts the surgeon into the best possible posture for which to conduct surgery and rest his own body while performing the surgery.
Also, it can produce and it can allow for longer, more intricate surgeries without taxing the surgeon to the point of exhaustion or ending the surgery prematurely because the surgeon himself can go no further with the surgery because of fatigue, pain and exhaustion.
